(19) United States
(12) Patent Application Publication
(10) Pub. No.: US 2007/0287171 A1
(43) Pub. Date: Dec. 13, 2007

(54) Process for production of proteins as soluble proteins

(75) Inventor: Satoshi Inouye, Yokohama-shi (JP)

Correspondence Address: HOGAN & HARTSON LLP;IP GROUP, COLUMBIA SQUARE, 555 THIRTEENTH STREET, N.W., WASHINGTON, DC, 20004, US

(73) Assignee: CHISSO Corporation

(21) Appl. No.: 11/808,092

(22) Filed: Jun. 6, 2007

(30) Foreign Application Priority Data
Jun. 7, 2006 (JP) 2006-158222

(51) Int. Cl. C07K 14/195; C07H 21/04; C12P 21/06; C12N 1/21

(52) U.S. Cl. 435/69.1; 435/320.1; 435/252.33; 530/350; 536/23.7

(57) ABSTRACT

The object of the invention is to produce a target protein as a soluble protein using a recombinant protein expression system. The invention provides a process for producing a target protein as a soluble protein, comprising the step of expressing a protein by using a polynucleotide containing, in order, a polynucleotide encoding a secretory signal peptide, a polynucleotide encoding a basic amino acid-rich polypeptide, and a polynucleotide encoding the target protein; and also provides an expression vector used for this purpose.

### CROSS REFERENCE TO RELATED APPLICATIONS

This application claims priority under 35 U.S.C. §119 to Japanese Patent Application No. JP 2006-158222, filed Jun. 7, 2006, which is expressly incorporated herein by reference in its entirety.

### BACKGROUND OF THE INVENTION

1. Field of the Invention

The invention relates to a process for producing a target protein as a soluble protein. More specifically, the invention relates to a process for producing a target protein as a soluble protein using a secretory signal peptide and a basic amino acid-rich peptide.

2. Related Art

Many recombinant protein expression systems have been developed to date, including recombinant protein expression systems in hosts such as bacteria, yeasts, insects, transgenic animals and transgenic plants, and cell-free translation systems. Of these, Escherichia coli is widely used as a heterologous protein expression system, both because E. coli cells can easily be grown to a high density and because of the advanced state of research on the host vector system.

However, when a target protein is expressed in these recombinant protein expression systems, it is often the case that, because the expressed protein does not fold accurately, the proper function of the protein cannot be expressed or insoluble aggregates called inclusion bodies are formed. In such cases, even if the inclusion bodies are denatured (solubilized) then refolded, there is no guarantee that a properly folded protein having functionality will be obtained. Moreover, even when it has been possible to obtain a properly functioning protein, the functional protein often cannot be obtained in an acceptable yield.

In spite of the above, a method for inhibiting the formation of inclusion bodies of expressed recombinant target proteins has yet to be established. An alternative approach currently being tried involves fusing the insoluble target protein with a highly soluble maltose-binding protein having a molecular weight of 40,000 or with glutathione S-transferase (GST), and thereby expressing the target protein as a soluble protein (see Fox, J. D. and Waugh, D. S., “Maltose-binding protein as a solubility enhancer,” Methods Mol. Bio., 205: 99-117 (2003); Ausubel, F. M. et al., eds., CURRENT PROTOCOLS IN MOLECULAR BIOLOGY, Vol. 2, 16.0.1 (1996)). However, there have been a number of problems with this approach, including the failure of the soluble protein to exhibit its proper activity and the fact that the target protein becomes insoluble when the maltose-binding protein or the GST is removed.

Renilla luciferin-binding protein (abbreviated below as “RLBP”) is known as a calcium-triggered luciferin-binding protein isolated from the sea pansy (Renilla reniformis). RLBP is a noncovalent complex of apoprotein (apoRLBP) and coelenterazine (luciferin) (J. Biol. Chem., 254, 769-780 (1979)). When RLBP binds to calcium ions, coelenterazine dissociates from RLBP. The dissociated coelenterazine is used in a luciferin-luciferase luminescent reaction by Renilla luciferase, Oplophorus luciferase or Gaussia luciferase in which coelenterazine serves as a luminescent substrate.

A process has been reported in which the E. coli L-asparaginase gene is fused to a PelB leader sequence and an N-terminal histidine hexamer tag, and recombinant E. coli L-asparaginase is thereby expressed into a culture filtrate from the bacterial cells, then purified by Ni—NTA affinity chromatography (Protein Express. Purif., 38, 29-36 (2004)).

### SUMMARY OF THE INVENTION

Under the above circumstances and in connection with the production of useful proteins, there exists a strong desire in industry for a way to produce a target protein as a soluble protein using a recombinant protein expression system. Such a need is also strongly felt in the field of research on protein function and structure.

It has been observed that, when an expression vector which contains a polynucleotide prepared by bonding a polynucleotide coding for a signal peptide and a polynucleotide coding for a basic amino acid-rich polypeptide with a polynucleotide coding for a target protein is used to bring about expression of the target protein in a gram-negative bacterium, the target protein is expressed as a soluble protein. The proper functionality of the target protein thus obtained has also been confirmed.

The invention includes:

(1) A process for producing a target protein as a soluble protein, including the step of expressing a protein by using a polynucleotide including, in order, a polynucleotide encoding a secretory signal peptide, a polynucleotide encoding a basic amino acid-rich polypeptide, and a polynucleotide encoding the target protein;

(2) The process of item (1) above, wherein protein expression is carried out in a host cell;

(3) The process of item (2) above, wherein the host cell is a gram-negative bacterium;

(4) The process of item (3) above, wherein the gram-negative bacterium is a bacterium of the genus Escherichia;

(5) The process of any one of items (1) to (4) above, wherein the secretory signal peptide is a secretory signal peptide from a gram-negative bacterium;

(6) The process of item (5) above, wherein the secretory signal peptide from a gram-negative bacterium is a secretory signal peptide from a facultative anaerobic bacillus;

(7) The process of item (5) above, wherein the secretory signal peptide from a gram-negative bacterium is a secretory signal peptide from at least one of the outer membrane protein A of Escherichia coli (OmpA) and a secretory signal peptide from cholera toxin from Vibrio cholerae;

(8) The process of any one of items (1) to (7) above, wherein the basic amino acid-rich polypeptide is a polypeptide composed of from approximately 5 to approximately 12 amino acid residues;

(9) The process of any one of items (1) to (8) above, wherein the basic amino acid-rich polypeptide has a basic amino acid content of at least approximately 60%;

(10) The process of items (1) to (9) above, wherein the basic amino acid in the basic amino acid-rich polypeptide is selected from the group of histidine, arginine and lysine;

(11) The process of items (1) to (9) above, wherein the basic amino acid-rich polypeptide is polyhistidine;

(12) The process of any one of items (1) to (11) above, wherein expression of the target protein is carried out using an expression vector comprising a polynucleotide encoding the target protein;

(13) A process for producing a target protein as a soluble protein, including the step of expressing a protein in a gram-negative bacterium by using a polynucleotide comprising a polynucleotide encoding a secretory signal peptide of the gram-negative bacterium, a polynucleotide encoding a polypeptide composed of from approximately 5 to approximately 12 basic amino acid residues, and a polynucleotide encoding the target protein;

(14) A process for producing a target protein as a soluble protein, including the step of expressing a protein in a genus Escherichia bacterium by using a polynucleotide including a polynucleotide encoding OmpA, a polynucleotide encoding polyhistidine, and a polynucleotide encoding the target protein;

(15) The process of any one of items (1) to (14) above, wherein the target protein is a heterologous protein;

(16) The process of any one of items (1) to (14) above, wherein the target protein is selected from the group of apoRLBP, apoaequorin, apoclytin, apoobelin and apomitrocomin;

(17) The process of item (16) above, wherein the target protein is apoRLBP;

(18) A process for producing apoRLBP, including the step of expressing a protein within a gram-negative bacterium by using a polynucleotide including a polynucleotide encoding a secretory signal peptide of the gram-negative bacterium, a polynucleotide encoding a polypeptide composed of from approximately 5 to approximately 12 basic amino acid residues, and a polynucleotide encoding apoRLBP;

(19) A process for producing apoRLBP, including the steps of expressing a protein within E. coli by using a polynucleotide including a polynucleotide encoding OmpA, a polynucleotide encoding polyhistidine, and a polynucleotide encoding apoRLBP; and accumulating the expressed protein in the periplasmic space of E. coli;

(20) A process for producing RLBP, including the step of contacting the apoRLBP produced by the process of any one of items (17) to (19) above with coelenterazine or a derivative thereof;

(21) A process for preserving coelenterazine or a derivative thereof, including the step of preparing RLBP by contacting the apoRLBP produced by the process of any one of items (17) to (19) above with coelenterazine or a derivative thereof;

(22) RLBP including apoRLBP produced by the process of any one of items (17) to (19) above and coelenterazine or a derivative thereof;

(23) An expression vector including (a) a first coding region which encodes a secretory signal peptide, (b) a second coding region which encodes a basic amino acid-rich polypeptide, and (c) at least one restriction enzyme site at which can be inserted a third coding region which encodes a target protein;

(23a) The expression vector of item (23) above, wherein the first coding region is effectively ligated to a promoter;

(23b) The expression vector of item (23) above, wherein the first coding region, the second coding region and the restriction enzyme site are located in the same reading frame;

(23c) The expression vector of item (23) above, wherein the second coding region is downstream from the first coding region, and the restriction enzyme site is downstream from the second coding region;

(23d) The expression vector of item (23) above, wherein the restriction enzyme site is a multicloning site;

(24) The expression vector of item (23) above, wherein the secretory signal peptide is a secretory signal peptide from a gram-negative bacterium;

(25) The expression vector of item (24) above, wherein the secretory signal peptide from a gram-negative bacterium is a secretory signal peptide from a facultative anaerobic bacillus;

(26) The expression vector of item (24) above, wherein the secretory signal peptide from a gram-negative bacterium is a secretory signal peptide from at least one of the outer membrane protein A of Escherichia coli (OmpA) and a secretory signal peptide from cholera toxin from Vibrio cholerae;

(27) The expression vector of any one of items (23) to (26) above, wherein the basic amino acid-rich polypeptide is a polypeptide composed of from approximately 5 to approximately 12 amino acid residues;

(28) The expression vector of any one of items (23) to (27) above, wherein the basic amino acid-rich polypeptide has a basic amino acid content of at least approximately 60%;

(29) The expression vector of any one of items (23) to (28) above, wherein the basic amino acid in the basic amino acid-rich polypeptide is selected from the group of histidine, arginine and lysine;

(30) The expression vector of any one of items (23) to (27) above, wherein the basic amino acid-rich polypeptide is polyhistidine;

(31) An expression vector including (a) a first coding region which encodes a secretory signal peptide from a gram-negative bacterium, (b) a second coding region which encodes a polypeptide of from approximately 5 to approximately 12 basic amino acid residues, and (c) at least one restriction enzyme site at which can be inserted a third coding region which encodes a target protein; and

(32) An expression vector including (a) a first coding region which encodes OmpA, (b) a second coding region which encodes polyhistidine, and (c) at least one restriction enzyme site at which can be inserted a third coding region which encodes a target protein.

The invention makes it possible, when a target protein is produced using a recombinant protein expression system, to produce the target protein as a soluble protein, thus eliminating the need to denature (solubilize) the target protein. As a result, the invention enables the target protein to be obtained efficiently and in a high yield. The invention is thus highly beneficial as a process for producing useful proteins and proteins intended for functional and structural analysis.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 shows the base sequence of the synthetic apoRLBP gene obtained in Reference Example 3, the restriction enzyme recognition sites, and the amino acid sequence of apoRLBP, (A) showing the restriction enzyme map, the shaded areas indicating loop regions of the EF hand motif, and (B) showing the base sequence of the apoRLBP gene, the boxed areas indicating loop regions of the EF hand motif;

FIG. 2 shows the apoRLBP expression vector piP-His-RLBP containing the OmpA signal peptide and a histidine hexamer obtained in Example 1, and also shows the apoRLBP expression vector piP-RLBP containing the OmpA signal peptide obtained in Reference Example 4, (A) showing the restriction enzyme map for piP-His-RLBP, and (B) showing the amino acid sequences of piP-His-RLBP and piP-RLBP, and the signal peptide sequence cleavage sites;

FIG. 3 shows the results of SDS-PAGE analyses of the recombinant apoRLBP obtained in Example 1, the recombinant RLBP obtained in Example 2, and the recombinant RLBP obtained in Reference Example 4. The samples of the respective lanes are as follows, (A) Lane 1: Protein molecular weight markers (Tefco): β-galactosidase (116,000), phospholipase B (97,400), bovine serum albumin (69,000), glutamate dehydrogenase (55,000), lactate dehydrogenase (36,500), carbonate dehydrogenase (29,000), trypsin inhibitor (20,100); Lane 2: Precipitate obtained by centrifuging an ultrasonicate of the cultured cells obtained in Example 1 (piP-His-RLBP/BL21, 40 μl); Lane 3: Supernatant obtained by centrifuging an ultrasonicate of the cultured cells obtained in Example 1 (piP-His-RLBP/BL21, 40 μl); Lane 4: Precipitate obtained by centrifuging an ultrasonicate of the cultured cells obtained in Reference Example 4 (piP-RLBP/BL21, 40 μl); Lane 5: Supernatant obtained by centrifuging an ultrasonicate of the cultured cells obtained in Reference Example 4 (piP-RLBP/BL2 1, 40 μl). (B) Lane 1: Protein molecular weight markers; Lane 2: Purified histidine hexamer-tagged apoRLBP (2.2 μg) obtained in Example 1; Lane 3: Purified histidine hexamer-tagged RLBP (3.5 μg) obtained in Example 2;

FIG. 4 shows the absorption spectra for the recombinant RLBP obtained in Example 3, both in the absence and the presence of calcium ions. The solid line indicates the absorption spectrum in the absence of calcium ions, the dashed line indicates the absorption spectrum in the presence of calcium ions.—The protein concentration was 0.45 mg/mL;

FIG. 5 shows the luminescence reaction in Example 4 between the recombinant RLBP obtained in Example 3 and Renilla luciferase. The insert is an enlargement of the region indicated by the arrow; and

FIG. 6 shows the expression vector piP-H6-M(11) obtained in Example 6.

### SEQUENCE TABLE FREE TEXT

The SEQ ID NOs in the sequence listing indicate the following sequences:

SEQ ID NO: 1 shows the amino acid sequence of apoRLBP;

SEQ ID NO: 2 shows the base sequence of synthetic DNA which encodes apoRLBP;

SEQ ID NO: 3 shows the base sequence of an oligonucleotide which encodes a sequence composed of six histidines used in Reference Example 2 and Example 6;

SEQ ID NO: 4 shows the base sequence of an oligonucleotide which encodes a sequence composed of six histidines used in Reference Example 2 and Example 6;

SEQ ID NO: 5 shows the base sequence of the primer used in Reference Example 3; and

SEQ ID NO: 6 shows the base sequence of the primer used in Reference Example 3.

### DESCRIPTION OF THE PREFERRED EMBODIMENTS

The invention provides a process for producing a target protein, which process includes the step of expressing a protein by using a polynucleotide containing a polynucleotide encoding a secretory signal peptide, a polynucleotide encoding a basic amino acid-rich polypeptide, and a polynucleotide encoding the target protein. The invention also provides an expression vector which can be used in such a process. By using the inventive process to express a target protein in a fused state with a secretory signal peptide and a basic amino acid-rich polypeptide, the target protein is prevented from misfolding and consequently forming inclusion bodies, thus enabling the target protein to be produced in a solubilized state as a protein which is correctly folded and functional.

The inventive process for producing a target protein and other aspects of the invention are described more fully below.

Process for Producing a Target Protein

The invention provides a process for producing a target protein, including the step of expressing a protein by using a polynucleotide including, in order, (1) a polynucleotide encoding a secretory signal peptide, (2) a polynucleotide encoding a basic amino acid-rich polypeptide, and (3) a polynucleotide encoding the target protein. The process is described in greater detail below.

(1) Secretory Signal Peptide

“Secretory signal peptide” refers to a peptide region which has the role of transporting a protein or polypeptide that has been bonded to the secretory signal peptide across a cell membrane. The amino acid sequence of such secretory signal peptides and the nucleic acid sequences encoding such peptides are familiar to, and have been reported in, the art to which the invention relates (see, e.g., von Heijine, G., Biochim. Biophys. Acra, 947: 307-333 (1988); von Heijine, G., J. Membr. Biol., 115: 195-201 (1990)).

Secretory signal peptides have an amino acid sequence made up of generally approximately 10 to approximately 50 amino acid residues, most (generally approximately 55% to approximately 60%) of which are hydrophobic. The secretory signal peptide used in the invention is preferably a secretory signal peptide obtained from a prokaryotic organism, more preferably from a gram-negative bacterium, even more preferably from a facultative anaerobic bacillus, and most preferably from a bacterium of the genus Escherichia, the genus Pseudomonas, the genus Salmonella or the genus Vibrio. Exemplary secretory signal peptides include those mentioned in, for example, von Heijine, G. and Abrahmsen, L., FEBS Lett., 244: 439-446 (1989); and Tjalsma, H. et al., Microbiol. Mol. Rev., 515-547 (2000). The secretory signal peptide from the outer membrane protein A of Escherichia coli (OmpA) (Ghrayeb, J. et al., EMBO J., 3: 2437-2442 (1984)) and the secretory signal peptide from cholera toxin obtained from Vibrio cholerae are especially preferred.

So long as the secretory signal peptide used in the invention has the ability to transport a protein or polypeptide bound to the secretory signal peptide across a cell membrane, the peptide may be a variant. Illustrative examples of such variants include peptides which have an amino acid sequence with, in the amino acid sequence of the secretory signal peptide, one or more deleted, substituted, inserted and/or added amino acid, and which have the ability to transport a protein or polypeptide bound to the secretory signal peptide across a cell membrane. Such peptides are exemplified by peptides which have an amino acid sequence wherein from approximately 1 to approximately 10, from approximately 1 to approximately 9, from approximately 1 to approximately 8, from approximately 1 to approximately 7, from approximately 1 to approximately 6 (from approximately 1 to several), from approximately 1 to approximately 5, from approximately 1 to approximately 4, from approximately 1 to approximately 3, approximately 1 or approximately 2, or approximately 1 amino acid residue in the amino acid sequence of the secretory signal peptide has been deleted, substituted, inserted and/or added, and which have the ability to transport a protein or polypeptide bound to the secretory signal peptide across a cell membrane. A smaller number of the above deleted, substituted, inserted and/or added amino acid residues is generally more preferable. Any two or more types of changes from among deletions, substitutions, insertions and additions may occur concurrently. Such peptides are exemplified by peptides having an amino acid sequence with at least approximately 80%, at least approximately 85%, at least approximately 90%, at least approximately 93%, at least approximately 95%, at least approximately 97%, at least approximately 98%, at least approximately 99%, at least approximately 99.5%, at least approximately 99.8% or at least approximately 99.9% identity to the amino acid sequence of the target protein, and having the ability to transport a protein or polypeptide bound to the secretory signal peptide across a cell membrane. A higher percent identity is generally more preferable.

The secretory signal peptide generally has a cleavage site where it is cleaved by signal peptidase in connection with transport across a cell membrane. The secretory signal peptide used in the invention need not have a signal peptidase cleavage site, although the existence of a cleavage site is preferred. The cleavage site is preferably one which can be cleaved by the secretory signal peptidase of the host (e.g., E. coli) used to express the target protein.

(2) Basic Amino Acid-Rich Polypeptide

The basic amino acid-rich polypeptide used in the invention may be any polypeptide which has a basic amino acid and which, when fused to the N-terminal side of the target protein and expressed, is capable of increasing the solubility of the target protein. Illustrative examples of the basic amino acid-rich polypeptide include polypeptides having an isoelectric point on the alkaline side of the physiological pH and a ratio of the number of basic amino acid residues with respect to all the amino acid residues making up the polypeptide (basic amino acid residue content) of approximately 30% or more. The basic amino acid residue content is preferably approximately 60% or more, more preferably approximately 80% or more, and most preferably approximately 100%.

The number of amino acid residues in the basic amino acid-rich polypeptide is preferably from approximately 3 to approximately 20, more preferably from approximately 5 to approximately 12, even more preferably from approximately 5 to approximately 8, and most preferably approximately 6.

“Basic amino acid” refers to an amino acid having a basic side chain. Examples of basic amino acids include histidine, arginine and lysine. The basic amino acid used in the invention is preferably histidine, arginine or lysine. Histidine is especially preferred.

The ratio of the number of histidine residues with respect to the basic amino acid residues present on the basic amino acid-rich polypeptide (histidine residue content) is preferably at least approximately 60%, more preferably at least approximately 80%, and most preferably approximately 100%.

The basic amino acid-rich polypeptide used in the invention is preferably a polyhistidine. The number of histidine residues on the polyhistidine is preferably from approximately 5 to approximately 12, more preferably from approximately 5 to approximately 8, and most preferably approximately 6 (histidine hexamer).

(3) Target Protein

No particular limitation is imposed on the target protein in the inventive production process. For example, even proteins which readily form inclusion bodies when expressed with a recombinant protein expression system are capable of being produced as soluble proteins, and thus may be advantageously used. The target protein may be an apoprotein; that is, the protein portion of a holoprotein. Illustrative examples of suitable apoproteins include apoRLBP (see, e.g., FEBS Lett., 268, 287-290 (1990)), apoaequorin (see, e.g., Proc. Natl. Acad. Sci. USA, 82, 3154-3158 (1985)), apoclytin (see, e.g., FEBS Lett., 315, 343-346 (1993)), apomitrocomin (see, e.g., FEBS Lett., 333, 301-305 (1993)), and apoobelin (see, e.g., Gene, 153, 273-274 (1995)). SEQ ID NO: 1 shows the amino acid sequence of apoRLBP.

Additional illustrative, though non-limiting, examples include proteins (viral antigens) such as capsid proteins, core proteins, proteases, reverse transcriptases and integrases encoded by pathogenic viral genomes such as hepatitis B viruses, hepatitis C viruses, HIV viruses and influenza viruses; the antibodies Fab and (Fab)₂; growth factors such as platelet-derived growth factor (PDGF), stem cell growth factor (SCF), hepatocyte growth factor (HGF), transforming growth factor (TGF), nerve growth factor (NGF), epidermal growth factor (EGF), fibroblast growth factor (FGF) and insulin-like growth factor (IGF); cytokinins such as tumor necrosis factor, interferon and interleukin; hematopoietic factors such as erythropoietin, granulocyte colony-stimulating factor, granulocyte macrophage colony-stimulating factor, macrophage colony-stimulating factor and thrombopoietin; peptide hormones such as luteinizing hormone-releasing hormone (LH-RH), thyrotropin-releasing hormone (TRH), insulin, somatostatin, growth hormones, prolactin, adrenocorticotropic hormone (ACTH), melanocyte-stimulating hormone (MSH), thyroid-stimulating hormone (TSH), luteinizing hormone (LH), follicle-stimulating hormone (FSH), vasopressin, oxytocin, calcitonin, parathyroid hormone (PTH), glucagon, gastrins, secretin, pancreozymin, cholecystokinin, angiotensin, human placental lactogen, human chorionic gonadotropin (HCG), caerulein and motilin; analgesic peptides such as enkephalin, endorphin, dynorphin and kyotorphin; enzymes such as superoxide dismutase (SOD), urokinase, tissue plasminogen activator (TPA), asparaginase and kallikrein; peptide neurotransmittors such as bombesin, neurotensin, bradykinin and substance P; and albumin, collagen, proinsulin, rennin and α₁-antitrypsin.

The target protein of the invention also encompasses variants of the above proteins. Illustrative examples of such variants include proteins which have an amino acid sequence with, in the amino acid sequence of the above proteins, one or more deleted, substituted, inserted and/or added amino acid, and which have an activity of the same nature as the target protein. Such proteins are exemplified by proteins which have an amino acid sequence wherein from approximately 1 to approximately 100, from approximately 1 to approximately 90, from approximately 1 to approximately 80, from approximately 1 to approximately 70, from approximately 1 to approximately 60, from approximately 1 to approximately 50, from approximately 1 to approximately 40, from approximately 1 to approximately 30, from approximately 1 to approximately 20, from approximately 1 to approximately 10, from approximately 1 to approximately 9, from approximately 1 to approximately 8, from approximately 1 to approximately 7, from approximately 1 to approximately 6 (from approximately 1 to several), from approximately 1 to approximately 5, from approximately 1 to approximately 4, from approximately 1 to approximately 3, approximately 1 or approximately 2, or approximately 1 amino acid residue in the amino acid sequence of the above protein has been deleted, substituted, inserted and/or added, and which have an activity of the same nature as the target protein. A smaller number of the above deleted, substituted, inserted and/or added amino acid residues is generally more preferable. Any two or more types of changes from among deletions, substitutions, insertions and additions may occur concurrently. In this specification, “apoRLBP” refers not only to the protein having the amino sequence indicated in SEQ ID NO: 1, but also to variants thereof.

The target protein of the invention also encompasses “partial peptides” of the target protein. Partial peptides of the protein are exemplified by partial peptides composed of a continuous amino acid sequence from part of the amino acid sequence of the target protein, and preferably have an activity of the same nature as the target protein. Illustrative examples include polypeptides having an amino acid sequence including at least approximately 20 amino acid residues, and preferably at least approximately 50 amino acid residues, in the amino acid sequence of the target protein. Preferably, these polypeptides contain an amino acid sequence which corresponds to the portion that takes part in the target protein activity. Partial peptides used in the invention may be modified by the deletion, addition, substitution or insertion of one or more (e.g., preferably about approximately 1 to approximately 20, more preferably about approximately 1 to approximately 10, and even more preferably about approximately 1 to approximately 5) amino acid residues in the amino acid sequence of the above polypeptide.

The partial peptides used in the invention may be employed also as antigens for antibody production.

(4) Polynucleotide Used in the Invention

Polynucleotides that may be used in the invention are any which include a base sequence encoding the above-described secretory signal peptide, basic amino acid-rich polypeptide or target protein, although DNA is preferred. Exemplary DNA includes genomic DNA, genomic DNA libraries, cellular or tissue cDNA, cellular or tissue cDNA libraries, and synthetic DNA. An example of a polynucleotide which encodes the target protein of the invention is apoRLBP-encoding synthetic DNA having the base sequence shown in SEQ ID NO: 2.

The vectors used in the libraries are not subject to any particular limitation, and may be, for example, bacteriophages, plasmids, cosmids or phagemids. Also, amplification may be carried out directly by a reverse transcription polymerase chain reaction (abbreviated below as “RT-PCR”) using a total RNA or mRNA fraction prepared from the above-mentioned cell or tissue.

The polynucleotide (DNA) used in the invention which includes a polynucleotide encoding a secretory signal peptide, a polynucleotide encoding a basic amino acid-rich polypeptide and a polynucleotide encoding a target protein may additionally have, between the polynucleotide encoding the target protein and the polynucleotide encoding the secretory signal peptide or the polynucleotide encoding the basic amino acid-rich polypeptide, a polynucleotide encoding a cleavable peptide linker. “Cleavable peptide linker” refers herein to a peptide sequence which is capable of being cleaved by an enzymatically or chemically cleaving substance. Many peptide sequences which are cleaved by enzymes (proteases) or chemical substances are known (see, e.g, Harlow and Lane, ANTIBODIES: A LABORATORY MANUAL (Cold Spring Harbor, N.Y.: Cold Spring Harbor Laboratory Press, (1988)); Walsh, PROTEIN BIOCHEMISTRY AND BIOTECHNOLOGY (West Sussex, England: John Wiley & Sons, Ltd., (2002)). “Cleaving substance” refers herein to a chemical substance or enzyme which recognizes a cleavage site on a polypeptide and splits the polypeptide into two polypeptides by cleaving a bond within the polypeptide. Examples of cleaving substances include chemical substances and proteases.

(5) Production of Target Protein

The target protein of the invention can be produced by using an expression vector having ligated thereto (introduced therein) a polynucleotide (DNA) (referred to in the description of target protein production that follows as the “polynucleotide used in the invention”) which includes, in order, a polynucleotide encoding a secretory signal peptide, a polynucleotide encoding a basic amino acid-rich polypeptide and a polynucleotide encoding a target protein to express the target protein, then isolating and purifying the target protein that has been formed. Expression of the target protein using the expression vector may be carried out in a protein expression system such as a host cell or a cell-free translation system. Expression of the target protein is preferably carried out in a host cell (transformant) that has been transformed by the introduction of the above expression vector. Production may be carried out by culturing the transformant under conditions which enable it to express the polynucleotide (DNA) used in the invention that has been ligated to (inserted onto) the introduced expression vector so as to cause the transformant to manufacture and accumulate the target protein, then isolating and purifying the target protein. It is preferable to use a gram-negative bacterium, and especially E. coli, as the host cell. When a gram-negative bacterium is used as the host cell, it is preferable for the target protein that has been expressed to be transported across the internal cell membrane, transported through the periplasmic space or across the outer membrane, and secreted into the culture supernatant by the secretory signal peptide. Here, the target protein of the invention may be a heterologous protein. As used herein, “heterologous protein,” refers to a protein which is not natively produced in the protein expressing system (e.g., the host cell).

Inventive processes for producing the target proteins are described more fully below.

Construction of Expression Vector:

The expression vector used in the invention contains the polynucleotide used in the invention. The recombinant vector of the invention may be obtained by ligating (inserting) the polynucleotide (DNA) used in the invention to a suitable vector. More specifically, the recombinant vector may be obtained by cleaving the purified polynucleotide (DNA) used in the invention with a suitable restriction enzyme, then inserting the cleaved polynucleotide to a restriction enzyme site or multicloning site on a suitable vector, and ligating the polynucleotide to the vector. The vector for inserting the polynucleotide used in the invention is not subject to any particular limitation, provided it can be replicated in the host. Vectors that may be used for this purpose include plasmids and bacteriophages. Illustrative examples of suitable plasmids include plasmids from E. coli (e.g., pUC8, pUC118, pUC119, pBR322 and pBR325). An example of a suitable bacteriophage is the λ phage.

The polynucleotide of the invention is generally ligated downstream from the promoter in a suitable vector so as to be expressible. The promoter used is preferably one that is capable of expression in the host. For example, if the host during transformation is a genus Escherichia bacterium, preferred promoters include the 1pp promoter, the Trp promoter, the T7 promoter, the lac promoter, the recA promoter and the λPL promoter.

In addition to the above, the recombinant vector used in the invention may be one which includes, if desired, a ribosome binding sequence (SD sequence), a selective marker and the like. Illustrative examples of selective markers include the dihydrofolate reductase gene, the ampicillin resistance gene and the neomycin resistance gene.

Preparation of Transformant:

The expression vector used in the invention is introduced into the transformant used in the invention so as to enable the polynucleotide (DNA) used in the invention to be expressed. The transformant can be created by introducing into a suitable host the expression vector, obtained as described above, which contains the polynucleotide (DNA) used in the invention. The host is not subject to any particular limitation, provided it is capable of expressing the polynucleotide (DNA) used in the invention. For example, it may be a gram-negative bacterium. Exemplary gram-negative bacteria include facultative anaerobic bacilli. Examples of suitable facultative anaerobic bacilli include bacteria of the genera Escherichia, Pseudomonas, and Salmonella. Bacteria of the genus Escherichia include E. coli. Bacteria of the genus Pseudomonas include P. aeruginosa. Bacteria of the genus Salmonella include S. enterica. Of the above, the host is preferably a gram-negative bacteria, more preferably a facultative anaerobic bacillus, even more preferably a bacterium of the genus Escherichia, and most preferably E. coli.

Introduction of the expression vector into the host and transformation thereby may be carried out by any of various ordinary methods. Examples of suitable methods for introducing the expression vector into the host cell include the calcium phosphate method (Virology, 52, 456-457 (1973)) and electroporation (EMBO J., 1, 841-845 (1982)). Examples of methods for transforming genus Escherichia bacteria include the methods described in Proc. Natl. Sci. USA, 69, 2110 (1972), and Gene, 17, 107 (1982). Methods for transforming genus Pseudomonas bacteria include, for example, electroporation. Methods for transforming genus Salmonella bacteria include, for example, electroporation.

A transformant created by transformation with an expression vector containing the polynucleotide (DNA) used in the invention can be obtained in this way.

Culturing the Transformant:

The transformant used in the invention may be cultivated by an ordinary method used for culturing hosts. With such cultivation, the target protein is produced by the transformant and accumulates in the periplasmic space or the culture broth.

The medium for culturing the transformant obtained using a genus Escherichia, Pseudomonas or Salmonella bacterium as the host may be a natural medium or a synthetic medium, provided it is a medium which contains the carbon sources, nitrogen sources, inorganic salts and other nutrients essential for growth of the transformant, and in which the transformant can be efficiently grown. Examples of carbon sources that may be used include carbohydrates such as glucose, fructose, sucrose and starch; organic acids such as acetic acid and propionic acid; and alcohols such as ethanol and propanol. Examples of nitrogen sources that may be used include ammonia, ammonium salts of inorganic or organic acids, such as ammonium chloride, ammonium sulfate, ammonium acetate and ammonium phosphate, other nitrogen-containing compounds, and also peptone, meat extract and corn steep liquor. Examples of inorganic salts include monobasic potassium phosphate, dibasic potassium phosphate, magnesium phosphate, magnesium sulfate, sodium chloride, ferrous sulfate, manganese sulfate, copper sulfate and calcium carbonate. If necessary, antibiotics such as ampicillin or tetracycline may be added to the medium during culturing. If the transformant to be cultured has been obtained by transformation with an expression vector using an induced promoter as the promoter, if necessary, the inducer may also be added to the medium. For example, isopropyl-β-D-thiogalactopyranoside (IPTG) may be added to the medium when culturing a transformant obtained by transformation with an expression vector using a Lac promoter, and indoleacrylic acid (IAA) may be added to the medium when culturing a transformant obtained by transformation with an expression vector using a trp promoter.

When the host is a bacterium of the genus Escherichia, Pseudomonas or Salmonella, incubation is generally carried out at approximately 15° C. to approximately 43° C. for approximately 3 to approximately 24 hours. If necessary, aeration and stirring may be applied.

Isolation and Purification of Target Protein:

The target protein of the invention can be obtained by isolating and purifying the target protein from the above-described culture. As used herein, “culture” refers to any one of the following: a culture broth, cultured bacteria, cultured cells, and the products obtained by disrupting cultured bacteria or cultured cells. An ordinary method may be used to isolate and purify the target.

Specifically, when the target protein accumulates in the periplasmic space, following the completion of cultivation, a target protein-containing extract may be obtained by an ordinary method such as osmotic shock. When the target protein accumulates in the culture broth, following the completion of cultivation, a culture supernatant containing the inventive protein may be obtained by using an ordinary method such as centrifugation or filtration to separate the culture supernatant from the bacteria or cells. When the target protein accumulates within cultured bacteria or within cultured cells, following the completion of cultivation, an extract of the target protein may be obtained by an ordinary method such as centrifugation or filtration after using an conventional technique (e.g, ultrasound, lysozymes, freezing and thawing) to disrupt the bacteria or cells.

Purification of the target protein present in the extract or culture supernatant obtained as described above may be carried out by an ordinary method of separation and purification. Examples of separation and purification methods that may be used include ammonium sulfate precipitation, gel filtration chromatography, ion-exchange chromatography, affinity chromatography, reversed-phase high-performance liquid chromatography, dialysis, and ultrafiltration, as well as suitable combinations thereof.

In cases where the target protein is an apoprotein, the holoprotein may be produced by a conventional method, such as by bringing the apoprotein obtained into contact with a non-protein component. For example, when the apoprotein obtained as described above is the apoprotein of a luciferin-binding protein which binds coelenterazine (e.g., apoRLBP, apoaequorin, apoclytin), the holoprotein (e.g., RLBP, apoRLBP, apoaequorin) of the luciferin-binding protein can be obtained by bringing the apoprotein into contact with coelenterazine or a derivative thereof (e.g., h-coelenterazine, e-coelenterazine, bis-coelenterazine). More specifically, RLBP may be prepared by the method described by, for example, H. Charbonneau and M. J. Cormier in “Ca²⁺-induced bioluminescence in Renilla reniformis. Purification and characterization of a calcium-triggered luciferin-binding protein,” J. Biol. Chem., 254, 769-780 (1979). Aequorin may be prepared by the method described by, for example, O. Shimomura and S. Inouye in “The in situ regeneration and extraction of recombinant aequorin from Escherichia coli cells and the purification of extracted aequorin,” Protein Expression and Purification, 16: 91-95 (1999).

Coelenterazine and coelenterazine derivatives are unstable in solution, but they are stable in the bound state within holoproteins of the luciferin-binding protein obtained by the production process of the invention. Therefore, the luciferin-binding protein apoprotein such as apoRLBP obtained by the inventive production process may be advantageously used for the stable preservation of luciferins such as coelenterazine and derivatives thereof.

In the specification, “holoproteins of luciferin-binding proteins” include holoproteins composed of the apoprotein of a luciferin-binding protein together with a luciferin (e.g., coelenterazine), and holoproteins composed of the apoprotein of a luciferin-binding protein together with a luciferin derivative (e.g., a coelenterazine derivative such as h-coelenterazine, e-coelenterazine or bis-coelenterazine). “RLBP” includes both holoproteins composed of apoRLBP together with coelenterazine, and holoproteins composed of apoRLBP together with a coelenterazine derivative.

2. Expression Vector of the Invention

The invention also provides an expression vector comprising: (a) a first coding region which encodes a secretory signal peptide, (b) a second coding region which encodes a basic amino acid-rich polypeptide, and (c) at least one restriction enzyme site at which can be inserted a third coding region which encodes a target protein. The expression vector of the invention may be advantageously used in the production of the above-described target protein by ligating (inserting) a polynucleotide which encodes the above-described target protein at (c) the at least one restriction enzyme site at which can be inserted a third coding region which encodes a target protein.

The signal peptide encoded by (a) the first coding region which encodes a secretory signal peptide is exemplified by the same peptides as are mentioned above for the above-described target protein production process.

The basic amino acid-rich polypeptide encoded by (b) the second coding region which encodes a basic amino acid-rich polypeptide is exemplified by the same polypeptides as are mentioned above for the above-described target protein production process.

The (c) at least one restriction enzyme site at which can be inserted the third coding region which encodes a target protein includes a polynucleotide having a restriction enzyme recognition site at which can be inserted the third coding region which encodes a target protein. The restriction enzyme site is not subject to any particular limitation, provided it is a site at which the third coding region which encodes the target protein can be inserted, although it is preferably a so-called multicloning site. Restriction enzyme sites such as multicloning sites are common knowledge and have been reported in the technical field of the invention (see, e.g., Yanisch-Perron, C., Vieira, J. and Messing, J., Gene, 33 (1985) 103-119, “Improved M13 phage cloning vectors and host strains: Nucleotide sequences of the M13mp18 and pUC19 vectors,” Gene, 33, 103-119 (1985)).

The target protein is exemplified by the same proteins as are mentioned above for the above-described target protein production process.

The expression vector of the invention is not subject to any particular limitation, so long as it can be replicated in the host. Exemplary expression vectors include plasmids and bacteriophages. Illustrative examples of plasmids include plasmids from E. coli (e.g., pUC8, pUC118, pUC119, pBR322 and pBR325). An example of a suitable bacteriophage is the λ phage. The host used for producing the above-described target protein is preferably a gram-negative bacterium, more preferably a facultative anaerobic bacillus, even more preferably a bacterium of the genus Escherichia, Pseudomonas or Salmonella, and most preferably a bacterium of the genus Escherichia. Therefore, the expression vector of the invention is preferably one which can be replicated in these bacteria, more preferably one which can be replicated in a bacterium of the genus Escherichia, and most preferably a plasmid from E. coli.

In the expression vector of the invention, the first coding region generally ligates effectively to a promoter. When the host is a bacterium of the genus Escherichia, the promoter used is preferably the 1pp promoter, Trp promoter, T7 promoter, lac promoter, recA promoter or λPL promoter. Given that the host used to produce the above-described target protein is preferably a gram-negative bacterium, more preferably a facultative anaerobic bacillus, even more preferably a bacterium of the genus Escherichia, and most preferably E. coli, the promoter used is preferably one which is capable of expression in these bacteria, more preferably one which is capable of expression in bacteria of the genus Escherichia, and most preferably one which is capable of expression in E. coli. More specifically, the use of the 1pp promoter, Trp promoter, T7 promoter, lac promoter, recA promoter or λPL promoter is preferred.

The above-described first coding region, second coding region and restriction enzyme site are arranged so as to be located within the same reading frame. The second coding region is preferably downstream from the first coding region, and the restriction enzyme site is preferably downstream from the second coding region.

Another preferred example of the inventive expression vector is one which incorporates, at the restriction enzyme site, a third coding region containing a polynucleotide which encodes the target protein.

Moreover, the inventive expression vector may additionally have, between at least one restriction enzyme site at which can be inserted a third coding region which encodes the above protein and the first coding region or the second coding region, a region which encodes a cleavable peptide linker. “Cleavable peptide linker” is used here in the same sense as that used above in connection with the target protein production process described above. The target protein encoded in the third coding region can be cut away by cleavage at the cleavage site of a peptide linker capable of being cleaved by treatment with a protease or a chemical substance.

Where no particular explanation is given in the preferred embodiments for working the invention or the examples of the invention, use will typically be made of the methods described in standard collections of protocols, such as J. Sambrook, E. F. Fritsch & T. Maniatis (Eds.), MOLECULAR CLONING, A LABORATORY MANUAL (3^{rd} edition) (Cold Spring Harbor, N.Y.: Cold Spring Harbor Press, 2001) and F. M. Ausubel, R. Brent, R. E. Kinston, D. D. Moore, J. G. Seidman, J. A. Smith, K. Struhl (Eds.), CURRENT PROTOCOLS IN MOLECULAR BIOLOGY (John Wiley & Sons, Ltd.), or modifications or variations thereof. When commercially available reagent kits and measurement equipment are used, unless noted otherwise herein, the protocols provided therewith will typically be followed.

The objects, features, advantages and ideas of the invention will be apparent to those skilled in the art from the description provided in the specification, and the invention will be readily practicable by those skilled in the art on the basis of the description appearing herein. The Description of the Preferred Embodiments and the Examples which show preferred modes for practicing the invention are included for the purpose of illustration and explanation, and are not intended to limit the scope of the claims. It will be apparent to those skilled in the art that various modifications may be made in how the invention is practiced based on described aspects in the specification without departing from the spirit and scope of the invention disclosed herein. Thus, it is intended that the invention covers the modifications and variations of this invention that come within the scope of any claims and their equivalents.

### EXAMPLES

Examples are given below to more fully illustrate the invention, and should not be construed as limiting the invention.

Materials and Procedures

First, the materials and procedures in the following examples are described.

(1) Materials

The following commercial products were used as the coelenterazines and coelenterazine derivatives: Coelenterazine (Chisso Corporation), h-Coelenterazine (Chisso Corporation), e-Coelenterazine (Wako Pure Chemical Industries, Ltd.), Bis-Coelenterazine (Chisso Corporation).

The recombinant Renilla luciferase from Renilla reniformis was prepared by a method described in the literature (see Biochem. Biophys. Res. Commun., 233, 249-353 (1997)). The following materials were all commercially available products: Chelate Sepharose Fast Flow and Sephadex G25 (superfine grade) (Amersham Bioscience); imidazole, dithiothreitol (DTT), ethylenediaminetetraacetic acid (EDTA), nickel sulfate hexahydrate (Wako Pure Chemical Industries).

(2) Mass Spectrometry

Measurement was performed by matrix-assisted laser desorption/ionization-time of flight mass spectrometry (MALDI-TOF-MS) on the autoflex™ (Bruker Daltonics) by the method described in Anal. Biochem., 316, 216-222 (2003) using sinapic acid (Aldrich-Sigma) as the matrix. The data were obtained in positive reflector mode. Time-mass conversions were carried out by external calibration using apomyoglobin (horse; m/z=16952.6) as the standard. Calculated values for molecular weight were obtained using the program ExPASy PeptideMass (http://www.expasy.ch/cgi-bin/peptide-mas.pl).

(3) Protein Concentration

The protein concentration was determined by the Bradford dye-binding assay (Anal. Biochem., 72, 248-254 (1976)) using a commercial kit (BioRad) and using bovine serum albumin (Pierce Biotechnology) as the standard substance. SDS-PAGE analysis was carried out under reducing conditions using a 12% separating gel (Tefco) by the LaemmLi method (Nature, 227, 680-658 (1970)).

### Reference Example 1

### Construction of the Apoaequorin Expression Vector piP-HE

The apoaequorin expression vector piP-HE having a sequence encoding OmpA but lacking a sequence encoding a basic amino acid-rich polypeptide was constructed by the method described in, for example, Proc. Natl. Acad. Sci USA, 82, 3154-3158 (1985); J. Biochem., 105, 473-477 (1989), and Japanese Patent Laid-open No. S63-102695. The specific procedure used was as follows.

(1) Construction of piQ8-HE

The EcoRI-HindIII portion of the high-copy cloning vector pUC8 was digested by the respective restriction enzymes, following which the EcoRI-HindIII fragment of aequorin cDNA obtained from the cDNA clone pAQ440 prepared by the method described in Japanese Patent Laid-open No. S61-135586 was subcloned to this portion, thereby constructing piQ8-HE.

(2) Construction of piP-HE

piQ8-HE was digested by ScaI-HindIII, following which a ScaI-HindIII fragment which contained lipoprotein promoter (1pp), the lac operator and the OmpA gene and which had been cut from pIN-III 113 OmpA-1 was inserted here, thereby constructing the expression vector piP-HE.

### Reference Example 2

### Construction of the Apoaequorin Expression Vector piP-His6-HE

The apoaequorin expression vector piP-His-HE, which includes sequences coding for OmpA and histidine hexamer, was constructed as follows.

Using piP-HEΔ2E obtained from the apoaequorin secretory expression vector piP-HE (prepared by the method described in Reference Example 1) by removing the EcoRi site on the carboxy-terminal side with the use of a Klenow fragment to fill in, oligonucleotides encoding sequences of six histidines (Eco-His6-Hind Linker: 5′-AAT-TCC-CAC-CAT-CAC-CAT-CAC-CAT-GGT 3′ (SEQ ID NO: 3), and Eco-His6-Hind Linker: 5′-AG-CTT-ACC-ATG-GTG-ATG-GTG-GG 3′ (SEQ ID NO: 4)) were inserted at the HindIII-EcoRI site on piP-HEΔ2E, thereby constructing the expression vector piP-His6-HE.

### Reference Example 3

### Design and Chemical Synthesis of Gene Coding for ApoRLBP

Because the cDNA for apoRLBP has not yet been isolated, the gene that codes for apoRLBP was chemically synthesized by oligonucleotide assembly using the PCR process.

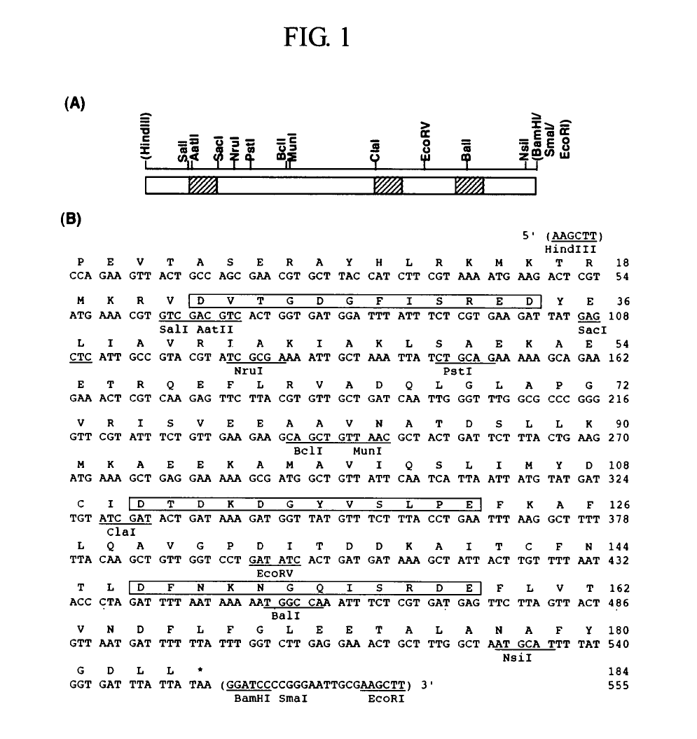

A gene coding for apoRLBP (184 amino acid sequences) was designed using DNASIS software Ver. 3.7 (Hitachi Software Engineering). At this time, codons preferred in E. coli were not used, and 11 restriction enzyme sites were introduced onto the 552-nucleotide sequence for apoRLBP. Oligonucleotides (40-mer×28, 35-mer×1) which replicate 20 nucleotides were synthesized on a 50 nmol scale by the phosphoamidate method using a Millipore DNA Synthesizer (model Expedite), purified by gel purification, and vacuum dried. Gene assembly was carried out using a PCR process (Gene, 164, 49-53 (1995)) described below. The dried oligonucleotides were re-suspended in distilled water at a concentration of approximately 3.3 μg/μl (250 mM), 1 μl of the respective internal oligonucleotide solutions were combined, and the mixture (0.4 μl) was added to 40 μl of a PCR reaction mixture containing 0.25 mM dNTP, 5 units of ExTaq polymerase (Takara Shuzo) and 4 μl of a 10× ExTaq buffer (buffer composition not shown on product insert). The PCR program involved carrying out 55 cycles, each consisting of 30 seconds at 94° C., 30 seconds at 50° C. and 60 seconds at 72° C. (Perkin Elmer). The assembly reaction mixture (2.5 μl) was used in amplification (30 cycles, each of 30 seconds at 94° C., 30 seconds at 50° C., and 60 seconds at 72° C.) by an outer primer set (3.3 μg): 15NL (5′ GGCAAGCTT-CCA-GAA-GTT-ACT-GCC-AGC-GAA-CGT-GCT-TAC-C 3′ (SEQ ID NO: 5), in which the HindIII site is underlined); and 33RL (5′GCCGGATCC-TTA-TAA-TAA-ATC-ACC-ATA-AAA-TGC-ATT-AGC-C 3′ (SEQ ID NO: 6), in which the BamHI site is underlined). The amplified fragments (approx. 550 base pairs) on 1.2% agarose gel were eluted with 6M NaI, and purified using a PCR purification kit (Qiagen). The separated fragments were digested with HindIII and BamHI, giving HindIII-BamHI fragments. The resulting HindIII-BamHI fragments of the synthetic apoRLBP gene corresponded to 184 amino acid residues, and had 11 restriction enzyme recognition sites among the 512 nucleotides (FIG. 1). The HindIII-BamHI fragment thus obtained was ligated to the HindIII/BamHI site of the expression vector pUC9-2 (Gene, 30, 247-250 (1984)), giving p92-RLBP. The DNA sequence was determined with the Applied Systems DNA Sequencer (models 377 and 310).

### Reference Example 4

### Expression of Recombinant ApoRLBP Having OmpA, but Lacking Basic Amino Acid-Rich Polypeptide

(1) Construction of piP-RLBP

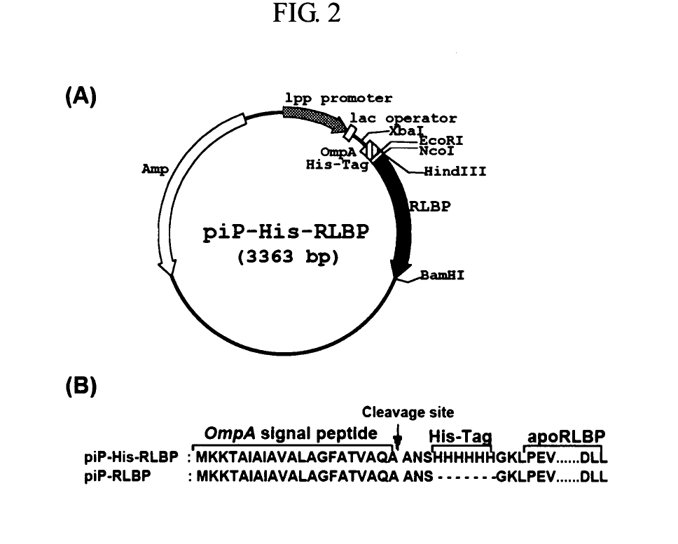

The expression plasmid piP-RLBP for apoRLBP having the OmpA signal peptide was constructed by replacing the HindIII-BamHI fragment of apoaequorin cDNA in the expression vector piP-HE obtained in Reference Example 1 with the HindIII-BamHI fragment of p92-RLBP obtained in Reference Example 3 (FIG. 2). The E. coli strain BL21 (Amersham Bioscience) was used as the host.

(2) Expression of Recombinant ApoRLBP in E. coli

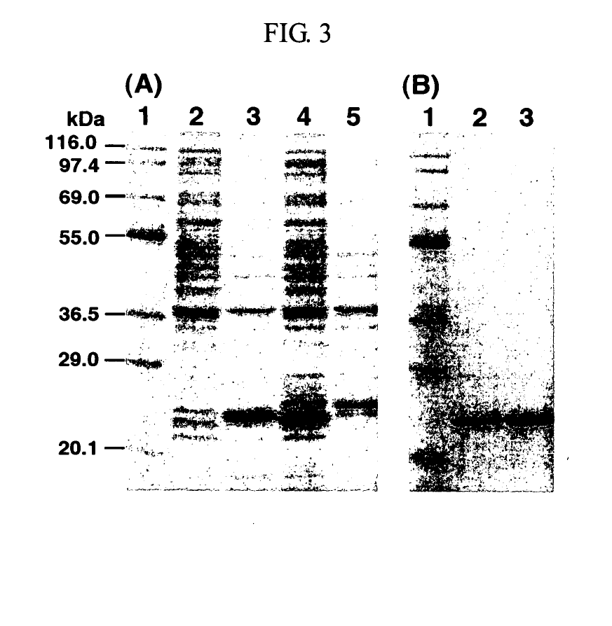

The bacterial strain containing the expression plasmid piP-RLBP that was obtained in (1) above was seed cultivated out in 5 mL of a Luria-Bertani (LB) medium containing ampicillin (50 μg/mL) at 30° C. for 16 hours, following which the seed culture was added to 80 mL of LB medium in a 500 mL Sakaguchi flask. After 16 hours of cultivation at 37° C., the cells were collected by 5 minutes of centrifugation at 5,000 g, suspended in 20 mL of 50 mM Tris-HCl (pH 7.6), and ultrasonically disrupted in ice using a Branson model 250 Sonifier. SDS-PAGE analysis confirmed the expression of apoRLBP lacking histidine. However, the expressed apoRLBP was present as inclusion bodies in the bacterial cells (FIG. 3A, Lane 4).

### Example 1

### Expression in E. coli of Recombinant ApoRLBP Containing OmpA and Histidine Hexamer

(1) Construction of piP-His-RLBP

The expression plasmid piP-His-RLBP for apoRLBP containing the OmpA signal peptide and a histidine hexamer was constructed (FIG. 2A) by replacing the HindIII-BamHI fragment of the apoaequorin cDNA in the expression vector piP-His6-HE obtained in Reference Example 2 with the HindIII-BamHI fragment of p92-RLBP obtained in Reference Example 3. The E. coli strain BL21 (Amersham Bioscience) was used as the host.

(2) Expression in E. coli of Recombinant ApoRLBP, and Purification

The bacterial strain containing the expression plasmid piP-His-RLBP that was obtained in (1) above was seed cultivated in 5 mL of a Luria-Bertani (LB) medium containing ampicillin (50 μg/mL) at 30° C. for 16 hours, following which the seed culture was added to 80 mL of LB medium in a 500 mL Sakaguchi flask. After 16 hours of cultivation at 37° C., the cells were collected by 5 minutes of centrifugation at 5,000 g, suspended in 20 mL of 50 mM Tris-HCl (pH 7.6) and ultrasonically disrupted in ice using a Branson model 250 Sonifier.

SDS-PAGE analysis confirmed expression of the recombinant apoRLBP as soluble protein using the expression vector piP-His-RLBP (FIG. 3A, Lane 3). Next, 20 mL of the supernatent obtained by centrifuging for 10 minutes at 12,000 g the ultrasonicate of the cultured cells was applied to a nickel chelate column (1.5×4 cm) equilibrated with 50 mM Tris-HCl (pH 7.6). The column was washed with 50 mL of 50 mM Tris-HCl (pH 7.6), following which the adsorbed protein was eluted in a stepwise manner with 20 mL each of 50 mM Tris-HCl (pH 7.6) containing 0.05 M, 0.1 M, 0.3 M, 0.5 M, and 1 M of imidazole. The histidine-tagged apoRLBP fractions were eluted with 0.1 to 0.3 M imidazole, and were subjected to SDS-PAGE analysis. The apoRLBP fractions were then combined, dialyzed with 4 liters of 50 mM ammonium bicarbonate (pH 8.3), and preserved at −80° C. The yield of the purified apoRLBP from 80 mL of cultured cells was 18.2 mg.

SDS-PAGE analysis under heat treatment and reducing conditions indicated that the purified apoRLBP had a purity of at least 95% and a molecular weight of 24 kDa (FIG. 3B, Lane 2). These results suggest that the amount of apoRLBP expressed in E. coli cells was at least 10% of the total protein extracted from the bacterial cells. This indicates that the inventive production process is able to produce a large amount of the target protein as soluble protein.

Mass spectrometry using MALDI-TOF-MS was carried out on the purified apoRLBP to ascertain that the OmpA signal peptide had been correctly cleaved and to confirm that the product was histidine hexamer-tagged apoRLBP. Mass values of m/z 21931.7 for [M+H]+ and of m/z 10965.9 for [M+2H]2+ were observed. These values were in close agreement with the calculated average mass of 21932.9 for histidine hexamer-tagged apoRLBP lacking the OmpA signal peptide. This result indicates that the OmpA signal peptide in histidine hexamer-tagged apoRLBP was correctly cleaved during migration from cytoplasm in the E. coli cells to the periplasmic space.

### Example 2

### Preparation of RLBP from ApoRLBP

The purified apoRLBP obtained in Example 1 (1 mg=46 nmol) was dissolved in 5 mL of 30 mM Tris-HCl (pH 7.6) containing 10 mM EDTA and 0.66 mM DTT, following which the conversion from apoRLBP to RLBP was begun by adding coelenterazine (20 μg=47 nmol). The mixture was left at rest for 16 hours at 4° C., then concentrated to 0.5 mL at 4° C. using a centrifugal filter unit (Amicon Ultra; molecular weight cutoff, 10,000), thereby giving a yellow-green solution. To remove unbound coelenterazine, this solution was applied to a Sephadex G-25 column (1×6 cm) equilibrated with 30 mM Tris-HCl (pH 7.6) containing 10 mM EDTA, and eluted with the same buffer. Fractions (0.5 mL) were collected at a flow rate of 0.5 mL per minute, and monitored for absorption at 280 nm and 446 nm. The main fractions of RLBP were eluted at from 0.5 mL to 1.0 mL. Recovery of the protein was 80%. The results of SDS-PAGE analysis under heat treatment and reducing conditions on the purified RLBP thus obtained are shown in Lane 3 of FIG. 3B. The purity of the purified RLBP was estimated, by comparison with absorption spectral data for native RLBP (J. Biol. Chem., 254, 769-780 (1979)), to be 95% or more.

This result indicates that the apoRLBP obtained in Example 1, like native RLBP, bonds with coelenterazine to form the holoprotein RLBP. Hence, the recombinant apoRLBP obtained in Example 1 was confirmed to be functional.

### Example 3

### Spectral Analysis of Recombinant RLBP

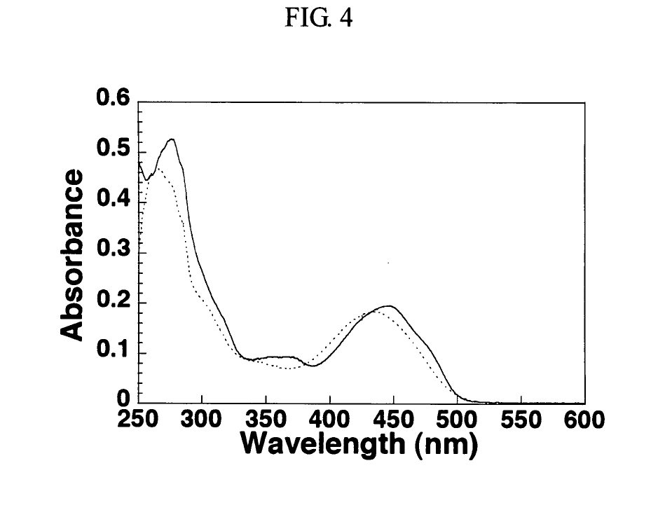

The absorption spectrum for the purified recombinant RLBP was measured in 20 mM Tris-HCl (pH 7.6) containing 2 mM EDTA or 10 mM CaCl₂ with a spectrophotometer (V-560, JASCO, Tokyo; bandpass, 0.5 nm; response, quick; scan rate, 100 nm/minute) at 25° C. and using a quartz cuvette (10 mm optical path). FIG. 4 shows the absorption spectra for recombinant RLBP in the presence and absence of calcium ions. The recombinant RLBP had two characteristic maximum absorptions: at 277 nm and at 446 nm, with a shoulder at 475 mm. This spectrum agrees substantially with the spectrum for native RLBP (J. Biol. Chem., 254, 769-780 (1979)). The absorption coefficients (ε) for recombinant RLBP at 277 nm and 446 nm were respectively 24,900 (E_{0.1%}=1.13) and 9,300 (E_{0.1%}=0.42). The 277 nm/446 nm absorption ratio for recombinant RLBP was 2.68, which is close to the 277 nm/446 nm ratio for native RLBP of between 2.7 and 2.8 (J. Biol. Chem., 254, 769-780 (1979)). In the presence of 10 mM of calcium ions, the 277 nm and 446 nm peaks for recombinant RLBP shifted to 265 nm and 434 nm, indicating properties similar to those of native RLBP (J. Biol. Chem., 254, 769-680 (1979)). The absorption peak at 420 nm (ε=9,300) for coelenterazine in the same buffer containing 10 mM EDTA shifted to 432 nm (ε=10,000) in the presence of 10 mM calcium ions. This absorption peak was close to the peak of 434 nm for recombinant RLBP in the presence of 10 mM calcium ions.

In addition, the fluorescence spectrum was measured using a Jasco FP-6500 W fluorescence spectrometer (fluorescence and excitation bandpass, 5 nm; response, 0.5 seconds; scan rate, 100 nm/minute). The fluorescence spectrum for RLBP excited at 446 nm shows, at one-half bandwidth=86 nm, a fluorescence peak at 530 nm, indicating a four-fold rise in the intensity of the fluorescence peak with the addition of calcium ions. These spectral change data indicate that recombinant RLBP has the same high-order structure as native RLBP, suggesting that it has folded correctly.

Judging from the absence of changes in the absorption spectrum, the solution of recombinant RLBP was stable for 6 months or more at 4° C. and −80° C. This indicates that, although a neutral or substantially neutral aqueous solution of coelenterazine generally has a half-life of about 3 days when stored at 4° C., stable, long-term storage is possible with the use of recombinant RLBP.

### Example 4

### Photoluminescence Reaction Between RLBP and Renilla Luciferase

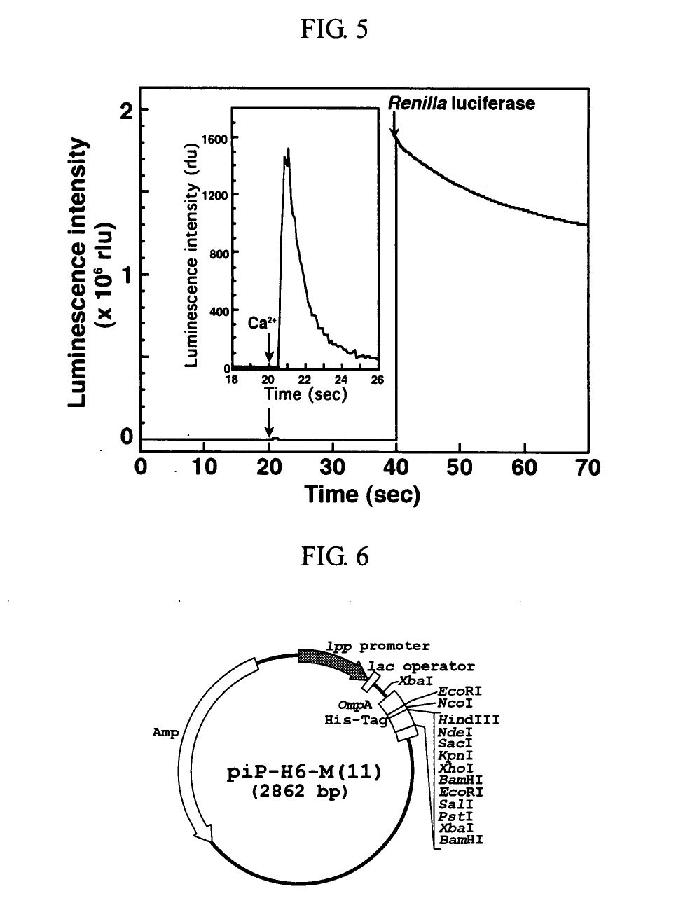

Renilla luciferase (0.08 μg) was added to a reaction mixture (100 μl) containing 50 mM of Tris-HCl (pH 7.6), 10 mM of CaCl₂ and 5 μg of the recombinant RLBP obtained in Example 3. The resulting luminescence activity was measured using a Luminometer (AB2200; Atto Corporation, Tokyo) equipped with a photomultiplier tube (R4220P, Hamamatsu Photonics), whereupon continuous luminescence was observed (FIG. 5). This data shows that the coelenterazine included within the RLBP molecule dissociated with the bonding of calcium ions to RLBP, and the dissociated coelenterazine became the substrate for the Renilla luminiferase and was oxidized by catalytic action, resulting in luminescence. This result indicates that calcium ions cause coelenterazine to dissociate from the RLBP obtained in Example 2 (the product of the apoRLBP obtained in Example 1 bonding with coelenterazine). As a result, the recombinant apoRLBP obtained in Example 1 and the recombinant RLBP obtained in Example 3 were confirmed to be functional.

### Example 5

### Preparation of RLBP from ApoRLBP and Coelenterazine Derivatives

Novel RLBPs were prepared in the same way as the method of preparing RLBP from apoRLBP and coelenterazine described in Example 2, but using coelenterazine derivatives. h-Coelenterazine, e-coelenterazine and Bis-coelenterazine were used as the coelenterazine derivatives, thereby giving the corresponding RLBPs; namely, h-RLBP, e-RLBP and Bis-RLBP. Table 1 shows the absorption spectral data, both in the absence and the presence of calcium ions (Ca²⁺), for apoRLBP, coelenterazine and the RLBPs obtained as described above. The absorption spectra were measured in the same way as in Example 3 in 50 mM Tris-HCl (pH, 7.6) containing 2 mM EDTA or 10 mM CaCl₂. Even when stored for 6 months or more at 4° C. and −80° C., the resulting RLBPs showed substantially no change in the absorption spectra. These results indicate that the recombinant RLBP solution is stable for at least 6 months at 4° C. and −80° C. That is, although coelenterazine derivatives, like coelenterazine, are unstable in solution, by using apoRLBP, they can be more stably preserved.

TABLE 1

| | | | εmax | | εmax | |
|---|---|---|---|---|---|---|
| | | λmax | (M⁻¹cm⁻¹): | λmax | (M⁻¹cm⁻¹): | (a)/ |
| | Ca²⁺ | (nm) | (a) | (nm) | (b) | (b) |
| ApoRLBP | − | 277 | 13,300 | | | |
| | + | 277 | 14,800 | | | |
| RLBP | − | 277 | 24,900 | 446 | 9,300 | 2.68 |
| | + | 265 | 19,700 | 434 | 8,900 | 2.21 |
| Coelenterazine | − | 259 | 25,700 | 420 | 9,300 | 2.76 |
| | + | 260 | 20,800 | 432 | 10,000 | 2.08 |
| h-RLBP | − | 274 | 22,700 | 450 | 8,800 | 2.59 |
| | + | 267 | 23,400 | 440 | 8,800 | 2.68 |
| e-RLBP | − | 281 | 21,500 | 458 | 8,200 | 2.63 |
| | + | 279 | 21,500 | 452 | 6,600 | 3.25 |
| Bis-RLBP | − | 265 | 20,300 | 448 | 8,100 | 2.51 |
| | + | 259 | 19,900 | 441 | 7,800 | 2.56 |

### Example 6

### Construction of Novel Basic Vector piP-H6-M(11) for Expressing a Secretory Protein Having an Amino-Terminal Histidine Sequence

The basic vector piP-H6-M(11) for expressing the target protein as soluble protein was constructed as follows. The starting vector piP-His6-HE was constructed according to the method described in Reference Example 2. In addition, a linker having a multicloning site (NcoI/HindIII/NdeI/SacI/KpnI/XhoI/BamHI/EcoRI/SalI/PstI/XbaI) was inserted at the HindIII-BamHI site on the piP-His6-HE vector, thereby constructing piP-H6-M(11). The basic vector piP-H6-M(11) was controlled by the lipoprotein promoter and the lactose operator in E. coli, and had an OmpA sequence for secretion, a sequence composed of six histidines, and a multicloning site (EcoRI/NcoI/HindIII/NdeI/SacI/KpnI/XhoI/BamHI/EcoRI/SalI/PstI/XbaI/BamHI) (FIG. 6).

### INDUSTRIAL APPLICABILITY

The inventive process for producing a target protein enables a target protein to be produced as a soluble protein, as a result of which there is no need for degrading (solubilizing) the target protein. The target protein can thus be efficiently obtained in a high yield by the inventive process. Accordingly, the process of the invention is highly beneficial for the production of proteins, including useful proteins and proteins for functional and structural analysis.

Moreover, the expression vector of the invention makes it possible to produce the target proteins as a soluble protein by inserting a gene coding for a target protein at a restriction enzyme site and thus allows the target protein to be expressed. Consequently, the invention is highly suitable for use in the production of desired proteins such as useful proteins.

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention

1. A process for producing a target protein as a soluble protein, comprising the step of expressing a protein by using a polynucleotide comprising a polynucleotide encoding a secretory signal peptide, a polynucleotide encoding a basic amino acid-rich polypeptide, and a polynucleotide encoding the target protein.

2. The process of claim 1, wherein protein expression is carried out in a host cell.

3. The process of claim 2, wherein the host cell is a gram-negative bacterium.

4. The process of claim 3, wherein the gram-negative bacterium is a bacterium of the genus Escherichia.

5. The process of claim 1, wherein the secretory signal peptide is a secretory signal peptide from a gram-negative bacterium.

6. The process of claim 5, wherein the secretory signal peptide from a gram-negative bacterium is a secretory signal peptide from a facultative anaerobic bacillus.

7. The process of claim 5, wherein the secretory signal peptide from a gram-negative bacterium is a secretory signal peptide from at least on of the outer membrane protein A of Escherichia coli (OmpA) and a secretory signal peptide from cholera toxin from Vibrio cholerae.

8. The process of claim 1, wherein the basic amino acid-rich polypeptide is a polypeptide composed of from approximately 5 to approximately 12 amino acid residues.

9. The process of claim 1, wherein the basic amino acid-rich polypeptide has a basic amino acid content of at least approximately 60%.

10. The process of claim 1, wherein the basic amino acid in the basic amino acid-rich polypeptide is selected from the group of histidine, arginine and lysine.

11. The process of claim 1, wherein the basic amino acid-rich polypeptide is polyhistidine.

12. The process of claim 1, wherein expression of the target protein is carried out using an expression vector comprising a polynucleotide encoding the target protein.

13. A process for producing a target protein as a soluble protein, comprising the step of expressing a protein in a gram-negative bacterium by using a polynucleotide comprising a polynucleotide encoding a secretory signal peptide of the gram-negative bacterium, a polynucleotide encoding a polypeptide composed of from approximately 5 to approximately 12 basic amino acid residues, and a polynucleotide encoding the target protein.

14. A process for producing a target protein as a soluble protein, comprising the step of expressing a protein in a genus Escherichia bacterium by using a polynucleotide comprising a polynucleotide encoding OmpA, a polynucleotide encoding polyhistidine, and a polynucleotide encoding the target protein.

15. The process of claim 1, wherein the target protein is a heterologous protein.

16. The process of claim 1, wherein the target protein is selected from the group of apoRLBP, apoaequorin, apoclytin, apoobelin and apomitrocomin.

17. The process of claim 16, wherein the target protein is apoRLBP.

18. A process for producing apoRLBP, comprising the step of expressing a protein within a gram-negative bacterium by using a polynucleotide comprising a polynucleotide encoding a secretory signal peptide of the gram-negative bacterium, a polynucleotide encoding a polypeptide composed of from approximately 5 to approximately 12 basic amino acid residues, and a polynucleotide encoding apoRLBP.

19. A process for producing apoRLBP, comprising the steps of:
expressing a protein within E. coli by using a polynucleotide comprising a polynucleotide encoding OmpA, a polynucleotide encoding polyhistidine, and a polynucleotide encoding apoRLBP; and
accumulating the expressed protein in the periplasmic space of E. coli.

20. A process for producing RLBP, comprising the step of contacting the apoRLBP produced by the process of claim 17 with coelenterazine or a derivative thereof.

21. A process for preserving coelenterazine or a derivative thereof, comprising the step of preparing RLBP by contacting the apoRLBP produced by the process of claim 17 with coelenterazine or a derivative thereof.

22. RLBP comprising apoRLBP produced by the process of claim 17 and coelenterazine or a derivative thereof.

23. RLBP comprising apoRLBP produced by the process of claim 18 and coelenterazine or a derivative thereof.

24. RLBP comprising apoRLBP produced by the process of claim 19 and coelenterazine or a derivative thereof.

25. An expression vector comprising:
(a) a first coding region which encodes a secretory signal peptide;
(b) a second coding region which encodes a basic amino acid-rich polypeptide; and
(c) at least one restriction enzyme site at which can be inserted a third coding region which encodes a target protein.

26. The expression vector of claim 25, wherein the secretory signal peptide is a secretory signal peptide from a gram-negative bacterium.

27. The expression vector of claim 26, wherein the secretory signal peptide from a gram-negative bacterium is a secretory signal peptide from a facultative anaerobic bacillus.

28. The expression vector of claim 26, wherein the secretory signal peptide from a gram-negative bacterium is a secretory signal peptide from at least one of the outer membrane protein A of Escherichia coli (OmpA) and a secretory signal peptide from cholera toxin from Vibrio cholerae.

29. The expression vector of claim 25, wherein the basic amino acid-rich polypeptide is a polypeptide composed of from approximately 5 to approximately 12 amino acid residues.

30. The expression vector of claim 25, wherein the basic amino acid-rich polypeptide has a basic amino acid content of at least approximately 60%.

31. The expression vector of claim 25, wherein the basic amino acid in the basic amino acid-rich polypeptide is selected from the group of histidine, arginine and lysine.

32. The expression vector of claim 25, wherein the basic amino acid-rich polypeptide is polyhistidine.

33. An expression vector comprising:
(a) a first coding region which encodes a secretory signal peptide from a gram-negative bacterium;
(b) a second coding region which encodes a polypeptide consisting of from approximately 5 to approximately 12 basic amino acid residues; and
(c) at least one restriction enzyme site at which can be inserted a third coding region which encodes a target protein.

34. An expression vector comprising:
(a) a first coding region which encodes OmpA;
(b) a second coding region which encodes polyhistidine; and
(c) at least one restriction enzyme site at which can be inserted a third coding region which encodes a target protein.


SEQUENCE LISTING (6 sequences)

SEQ ID NO: 1 - protein, 184 aa, Renilla reniformis
PEVTASERAYHLRKMKTRMKRVDVTGDGFISREDYELIAVRIAKIAKLSAEKAEETRQEFLRVADQLGLAPGVRISVEEAAVNATDSLLKMKAEEKAMAVIQSLIMYDCIDTDKDGYVSLPEFKAFLQAVGPDITDDKAITCFNTLDFNKNGQISRDEFLVTVNDFLFGLEETALANAFYGDLL

SEQ ID NO: 2 - DNA, 585 bp, Artificial
  Synthetic gene of apoRLBP
  CDS (7)..(561)
aagcttccagaagttactgccagcgaacgtgcttaccatcttcgtaaaatgaagactcgtatgaaacgtgtcgacgtcactggtgatggatttatttctcgtgaagattatgagctcattgccgtacgtatcgcgaaaattgctaaattatctgcagaaaaagcagaagaaactcgtcaagagttcttacgtgttgctgatcaattgggtttggcgcccggggttcgtatttctgttgaagaagcagctgttaacgctactgattctttactgaagatgaaagctgaggaaaaagcgatggctgttattcaatcattaattatgtatgattgtatcgatactgataaagatggttatgtttctttacctgaatttaaggcttttttacaagctgttggtcctgatatcactgatgataaagctattacttgttttaataccctagattttaataaaaatggccaaatttctcgtgatgagttcttagttactgttaatgattttttatttggtcttgaggaaactgctttggctaatgcattttatggtgatttattataaggatccccgggaattgcggaattc

SEQ ID NO: 3 - DNA, 27 bp, Artificial
  Oligonuculeotide encoding Histidine hexamer
aattcccaccatcaccatcaccatggt

SEQ ID NO: 4 - DNA, 22 bp, Artificial
  Oligonucleotide encoding Histidine hexamer
agcttaccatggtgatggtggg

SEQ ID NO: 5 - DNA, 40 bp, Artificial
  Primer
ggcaagcttccagaagttactgccagcgaacgtgcttacc

SEQ ID NO: 6 - DNA, 40 bp, Artificial
  Primer
gccggatccttataataaatcaccataaaatgcattagcc